(19) United States
(12) Patent Application Publication
(10) Pub. No.: US 2003/0225437 A1
(43) Pub. Date: Dec. 4, 2003

(54) Device for retaining material

(76) Inventor: Patrick J. Ferguson, Portland, OR (US)

Correspondence Address: KOLISCH HARTWELL, P.C., 520 S.W. YAMHILL STREET, SUITE 200, PORTLAND, OR, 97204, US

(21) Appl. No.: 10/407,843

(22) Filed: Apr. 4, 2003

Related U.S. Application Data
(60) Provisional application No. 60/370,531, filed on Apr. 4, 2002.

(51) Int. Cl. A61B 17/06

(52) U.S. Cl. 606/222

(57) ABSTRACT

A stopple for retaining material within a hollow needle/push rod assembly is made of a synthetic absorbable material and is configured to fit snugly within the hollow needle of the needle/push rod assembly. The stopple is typically circular in shape, having a diameter that is substantially similar to the inner diameter of the hollow needle. The stopple prevents inadvertent ejection of material stored in the hollow needle/push rod assembly.

### CROSS-REFERENCE TO RELATED APPLICATIONS

This application claims priority to U.S. Provisional Patent Application Serial No. 60/370,531, filed Apr. 4, 2002 and entitled “A Device for Retaining Material”, which is hereby incorporated by reference.

### TECHNICAL FIELD

The present invention relates generally to surgical tools and more particularly to a stopple useful for retaining material within hollow needle/push rod assemblies.

### BACKGROUND AND SUMMARY OF THE INVENTION

Radiation therapy has been useful for treating individuals afflicted with cancer since the early 20^{th} century. Brachytherapy is an advanced form of radiation treatment that is used for treating prostate cancer. Typically, radioactive seeds or sources are placed within or near a tumor, giving a high radiation dose to the tumor while reducing the dose to surrounding healthy tissues in the body.

Hollow needle/push rod assemblies have become increasingly popular surgical tools for the delivery of seeds to tumors during Brachytherapy. Typically, the radioactive seeds are loaded into the hollow needle's cylindrical cavity, adjacent the push rod. The seeds are often interspersed between synthetic absorbable spacers. The spacers are of the same size and shape as the seeds, both typically being cylindrical with rounded ends. Once loaded, the needle/push road assembly is inserted into the tumor by a surgeon and the seeds are implanted by retracting the needle over the push rod, thereby ejecting the seeds from the hollow needle into the tumor. Alternatively, the seeds and spacers may be preloaded into a synthetic absorbable carrier, which is useful for retaining the seeds and spacers in a predefined location within living tissue. The carrier is then implanted in a tumor using a hollow needle/rod assembly as described above.

The use of hollow/needle push rod assemblies requires that the needle tip be unsealed so that the seeds can be easily loaded into the needle cavity. Furthermore, the seeds and spacers are designed to easily travel within the needle cavity so that they can be deployed quickly and efficiently into the desired tissue. Thus, the seeds and spacers are prone to being inadvertently ejected from the hollow needle tip during operation of the needle assembly. This poses a health risk to both the patient and others in proximity to surgical procedure, any one of whom might accidentally be exposed to radiation emitted by inadvertently ejected seeds. Therefore, it is desirable to improve the safety of Brachytherapy by preventing undesired seed ejection.

### SUMMARY OF THE INVENTION

A stopple is provided for retaining material within a hollow needle/push rod assembly. The stopple is typically composed of a synthetic absorbable material and is configured to fit snugly within the hollow needle of the needle/push rod assembly. The stopple is typically circular in shape, having a diameter that is substantially similar to the inner diameter of the hollow needle. The stopple is further configured to prevent inadvertent ejection of material stored in the hollow needle/push rod assembly.

### BRIEF DESCRIPTION OF THE DRAWINGS

FIG. 1 is an enlarged cross-sectional view of a circular stopple, shown adjacent to a Brachytherapy element, according to one embodiment of the present invention.

FIG. 2 is a partial cross-sectional view of a hollow needle/push rod assembly including seeds, spacers and a stopple located at the distal end of the needle, according one embodiment of the present invention.

FIG. 3 is a cross-sectional view of a carrier housing radioactive seeds, spacers, and a stopple, according to one embodiment of the present invention.

FIG. 4 is a partial cross-sectional view of a hollow needle/push rod assembly including the carrier shown in FIG. 3, according to one embodiment of the present invention.

### DETAILED DESCRIPTION OF THE PREFERRED EMBODIMENTS

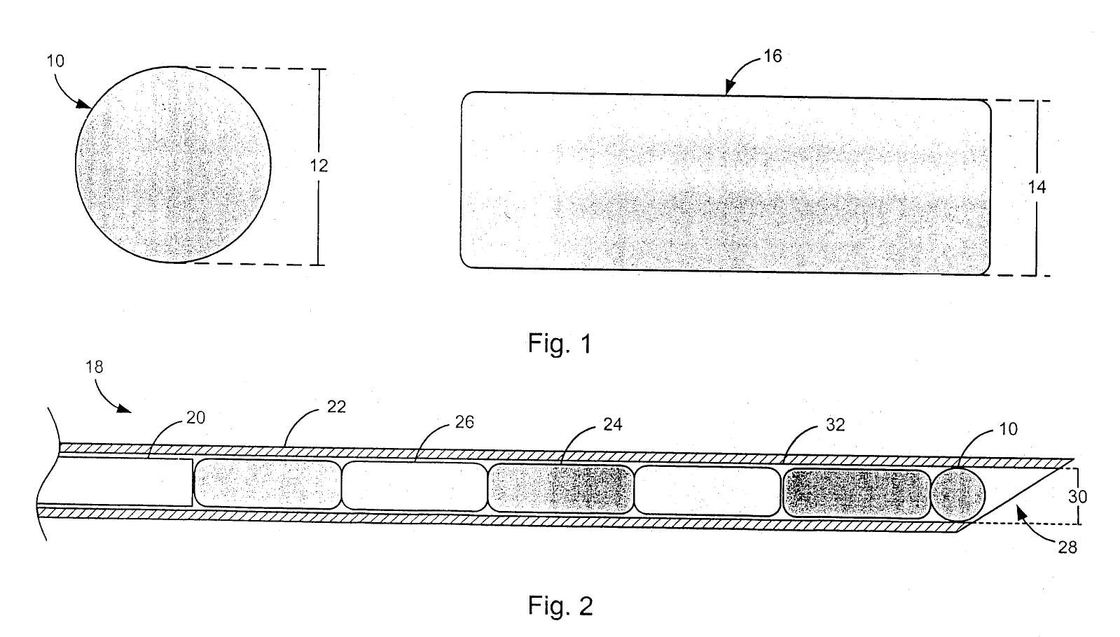

Referring initially to FIG. 1, one embodiment of a stopple useful for retaining material within a hollow needle/push rod assembly is generally shown at 10. The stopple has a body 10 that is typically circular in shape, having a diameter 12 that is greater than diameter 14 of Brachytherapy element 16. Body 10 is also referred to herein as the stopple. Brachytherapy element 16 is typically a radioactive seed or synthetic absorbable spacer useful in the treatment of prostate cancer. Typically, diameter 12 is approximately 0.1 mm to 100 mm, and is preferably 0.2 mm.

Stopple 10 is typically composed of at least One synthetic organic material such polylactic acid and/or its derivatives. In a preferred embodiment of the present invention, the composition of stopple 10 includes approximately 5-100% polyglycolic acid and 5-95% lactide. In a particularly preferred embodiment of the present invention, stopple 10 is composed of 90% polyglycolic acid and 10% lactide. In another preferred embodiment, stopple 10 is composed of 100% polyglycolic acid. In yet another preferred embodiment, stopple 10 is composed of 100% polydioxanone. Stopple 10 is typically manufactured in the same manner as the synthetic absorbable spacer described above using methods well known in the art.

Referring next to FIG. 2, a partial cross-sectional view of a hollow needle/push rod assembly is generally shown at 18. Assembly 18 typically includes push rod 20 and hollow needle 22, and is configured to deposit material in living tissue. Assembly 18 is also typically configured to retain at least one spacer 24 and at least one radioactive seed 26, and may retain up to eight seeds, or seeds and spacers. Spacers 24 are typically made of a synthetic absorbable material. In a preferred embodiment of the present invention, spacers 24 and stopple 10 are composed of the same synthetic absorbable material. Seeds 26 are typically composed of iodine-125, which has a half-life of 60 days, and are typically of the same size and shape as spacers 24. Spacers 26 and seeds 24 are typically arranged in a linear fashion within needle 22, along their longitudinal axis. Assembly 18 is further configured to retain stopple 10 at the distal end 28 of hollow needle 22.

Stopple 10 is typically configured to prevent inadvertent ejection of spacers 24 and/or seeds 26 from assembly 18. Typically, diameter 12 of stopple 10 is greater than the diameter of both spacers 24 and seeds 26 and is substantially similar to inner diameter 30 of needle 22. Frictional force applied by stopple 10 to inner surface 32 of needle 22 prevents stopple 10 from being dislodged from the needle, thus retaining the seeds and spacers. Typically stopple 10 resists applied forces of approximately 0.2 up to 4 lbs./in.². In a preferred embodiment of the present invention, stopple 10 is configured to resist an applied force of up to 0.8 lbs./in.². Exceeding the applied force is achieved by manually pushing push rod 20 along the longitudinal axis of needle 22, towards the distal end of the needle. Upon exceeding the applied force stopple 10, spacers 24 and seeds 26 are ejected from the needle.

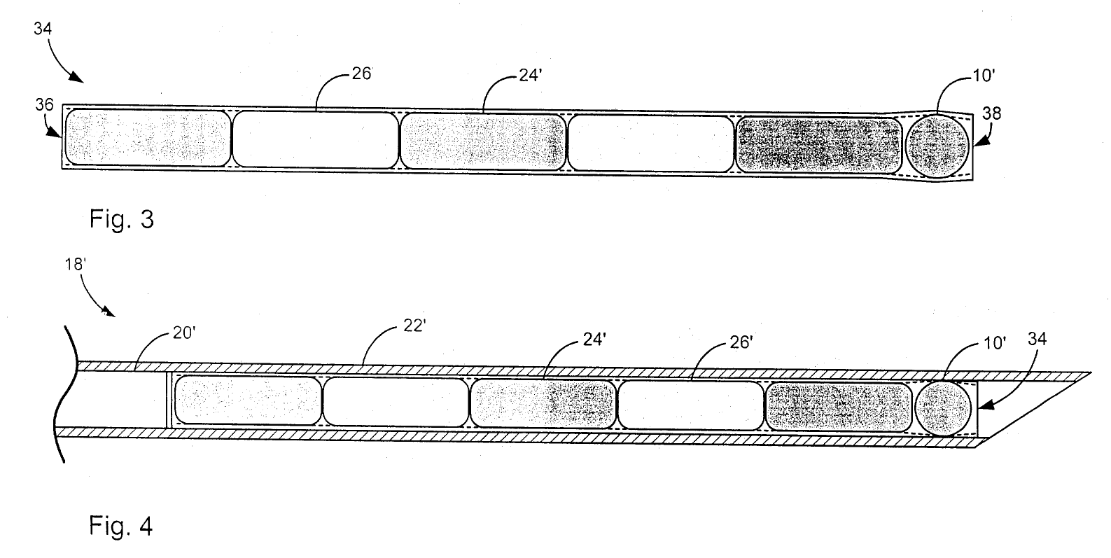

Referring next to FIG. 3, a carrier housing a stopple according to one embodiment of the present invention is shown generally at 34. Typically, carrier 34 is cylindrical in shape and has first and second ends 36 and 38, respectively. Carrier 34 is typically configured to retain spacers 24′, seeds 26′, and at least one stopple 10′. Stopple 10′ is typically configured to have a diameter that is greater than the diameter of the seeds and spacers. In one embodiment of the present invention, stopple 10′ is configured such that its diameter is slightly greater than the diameter of carrier 34, causing carrier 34 to be slightly deformed upon insertion of stopple 10′ into second end 38. Stopple 10′ is typically configured to prevent spacers 24′ and seeds 26′ from inadvertently falling out of second end 38.

Referring next to FIG. 4, a partial cross-sectional view of a hollow needle/push rod assembly according to another embodiment of the present invention is generally shown at 18′. Assembly 18′ typically includes push rod 20′ and hollow needle 22′, and is configured to retain carrier 34 (shown in FIG. 3). Similar to stopple 10 described above, stopple 10′ is configured to resist force applied along the longitudinal axis of needle 22′ that would otherwise dislodge spacers 24′ and seeds 26′. Carrier 34 is typically configured to be ejected from needle 22′ upon a force applied to push rod 20′ that is greater than the frictional force applied to inner surface 30′ by stopple 10′, as desired.

The above-described stopples 10, 10′ prevent inadvertent seed and spacer ejection during operation of needle/push rod assemblies for Brachytherapy and therefore reduce the health risks associated with handling radioactive seeds.

Although the invention has been disclosed in its preferred forms, the specific embodiments thereof as disclosed and illustrated herein are not to be considered in a limiting sense, because numerous variations are possible. The subject matter of the invention includes all novel and non-obvious combinations and subcombinations of the various elements, features, functions, and/or properties disclosed herein.

I claim:

1. A stopple for retaining material within a hollow needle/push rod assembly, comprising:
a body made from a synthetic absorbable material, configured to fit snugly within the hollow needle of the needle/push rod assembly, and constructed to prevent inadvertent ejection of material stored in the hollow needle/push rod assembly.
